(19) United States
(12) Patent Application Publication
(10) Pub. No.: US 2003/0082610 A1
(43) Pub. Date: May 1, 2003

(54) Use of a gene change in gene GNB3 coding for the B3 subunit of human G proteins

(75) Inventors: Winfried Siffert, Essen (DE); Hans Hauner, Neuss (DE)

Correspondence Address: NIXON PEABODY, LLP, 8180 GREENSBORO DRIVE, SUITE 800, MCLEAN, VA, 22102, US

(73) Assignee: SniP Biotech GmbH & Co. KG, Essen (DE)

(21) Appl. No.: 10/244,592

(22) Filed: Sep. 17, 2002

(30) Foreign Application Priority Data
Sep. 17, 2001 (DE) 101 45 814.2

(51) Int. Cl. C12Q 1/68; A61K 38/17; A61K 31/137

(52) U.S. Cl. 435/6; 514/12; 514/649

(57) ABSTRACT

The invention relates to sequence variants of the human gene GNB3 which codes for the Gβ3 subunit of human G proteins, and their use to predict the success of pharmacological and non-pharmacological measures for weight reduction within the framework of treatment of overweight and adiposity conditions.

### BACKGROUND OF THE INVENTION

1. Field of the Invention

The invention relates to sequence variants of the human gene GNB3 which codes for the Gβ3 subunit of human G proteins, and their use to predict the success of pharmacological and non-pharmacological measures for weight reduction within the framework of treatment of overweight and adiposity.

2. Description of Related Art

Adiposity and overweight are increasing in epidemic proportions worldwide. In addition to aesthetic-cosmetic problems, they contribute to formation of diverse diseases, including, not only for example, high blood pressure and stroke, but also cancers. It is generally known that different individuals react quite differently to weight reduction measures. It is likewise known that overweight and adiposity are based on the interaction of environmental factors and behavior with numerous different gene polymorphisms. Many individuals can reduce relatively easily within the framework of diets and structured weight reduction measures, while others have major problems in reducing increased body weight and body weight. These people often require, in addition to non-pharmacological measures, drugs to be able to reach the goal of long-term weight reduction.

Pharmacological and non-pharmacological measures are both commonly used for weight reduction. Non-pharmacological measures include increasing body activity (exercise) with a simultaneously reduced calorie intake. A host of food substitutes can be administered for this purpose, for example diet drinks, swelling substances which intensify the feeling of satisfaction. In addition, it is possible to carry out weight-reducing measures within structured weight reduction programs, for example, Optifast™ and Weight Watchers™. In certain programs, so-called “therapeutic fasts” are used, during with especially dramatic weight reduction occurs in many individuals. These measures can be supported by certain drugs. Many of these drugs such as Sibutramine (commercial name Meridia™, Reductil™) reduce the feeling of hunger and thus facilitate dieting. Sibutramine is a centrally acting reuptake inhibitor of the neurotransmitters serotonin and noradrenalin. This substance provides an increased feeling of fullness and thus leads to lower food intake. Other substances such as orlistat (commercial name for example Xenical™) reduce the absorption of fats from the supplied food (A. Ballinger, Orlistat in the treatment of obesity, Expert Opinion, Pharmacotherapy. 1 (4) 841-847, 2000). Likewise, a reduction of fat absorption over the long term ultimately leads to weight reduction. In the future, a host of other drugs which can similarly be used in the treatment of adiposity will come onto the market. These drugs can fundamentally act in various ways. They can reduce the feeling of hunger, directly stimulate lipolysis or influence thermoregulation such that the basal metabolic rate is increased. In addition, drugs can be used which delay the emptying of the stomach.

Most drugs activate or inhibit heptahelical receptors in the cell membrane with intracellular action mediated by heterotrimeric G proteins. Other drugs, such as sibutramine, increase the endogenous concentration of hormones and/or neurotransmitters with action mediated by G proteins (W. P. James et al., Effect of sibutramine on weight maintenance after weight loss: a randomized trial. STORM Study Group; Sibutramine Trial of Obesity Reduction and Maintenance,. Lancet 356 (9248): 2119-2125, 2000). These G proteins consist of different α, β, and γ subunits. After activation of the α subunit, it releases GDP in exchange against intracelluar GDP. The α and βγ subunits disassociate from one another and can then control intracellular effectors, for example ion channels, phospholipases, adenylylcyclases, PT-3 kinase and many more (H. R. Bourne, How receptors talk to trimeric G proteins. Current. Opinion, Cell Biology 9 (2): 134-142, 1997).

Based on the key function of the G proteins in cell activation, it can be expected that mutations in genes which code for G proteins have a lasting effect on the activatability of cells.

In earlier research a C825T polymorphism was found in gene GNB3 which codes for the Gβ3 subunit of G proteins (DE 196 19 366 A1). The 825T allele is associated with alternative splicing of the gene. In 825T allele carriers increased activatability of G proteins is found; this is expressed among others in increased responsiveness to a series of drugs. They include, for example, antidepressants (P. Zill et al., Evidence for an association between a G-protein beta3-gene variant with depression and response to antidepressant treatment, Neuroreport 11 (9): 1893-1897, 2000), diuretics (S. T. Turner et al., C825T polymorphism of the G protein beta(3) subunit and antihypertensive response to a thiazide diuretic, Hypertension 37 (2 part 2): 739-743, 2001) and α2 adrenoceptor agonists (D. Barmgart et al., G. Protein beta3 subunit 825T allele and enhanced coronary vasoconstriction on alpha(2) adrenoceptor activation, Circ. Res. 85 (10): 965-969, 1999). Accordingly, it can also be expected that 825T allele carriers react to an increased degree to endogenous released hormones and transmitters. It is generally known that dramatic withholding of food, for example in therapeutic fasts, leads to a change of the concentration of numerous hormones and transmitters. In particular, a dramatic increase of catecholamines is observed, to which the 825T allele carriers react especially strongly.

In gene GNB3 there is a series of other genetic polymorphisms. They include for example

a C1429T polymorphism

a G(−350)A polymorphism

a G1906T polymorphism in intron 4

a G2906A polymorphism in intron 5

an A3382C polymorphism in intron 9

a G5177A polymorphism in intron 9

a G5249A polymorphism in intron 9

a CACA insertion/deletion polymorphism in intron 10

Most of these alleles are in coupling disequilibrium among one another and with the C825T polymorphism so that in fact the following haplotypes are most often observed:

“C-haplotype”

Comprising alleles:

(−350)G, 3882A, 825C, 5249G, without the CACA insert in intron 10

“T-haplotype”

Comprising alleles:

(−350)A, 3882C, 825T, 5249A, with the CACA insert in intron 10

Thus these gene variants can be used just like the C825T polymorphism to predict strong or weak activatability of G proteins.

### SUMMARY OF THE INVENTION

The object of the current invention is to provide, through gene research, suitable genetic markers to assist in identifying and tailoring therapies for weight reduction. In this way, it is possible to avoid unnecessarily treating individuals who do not need drugs for weight reduction with drugs. Conversely, individuals can be identified who would benefit from drug therapy as part of a weight reduction program. It is described below how the use of a gene change in gene GNB3 is outstandingly well suited for predicting the success of pharmacological and non-pharmacological measures for weight reduction.

### BRIEF DESCRIPTION OF THE DRAWINGS

FIG. 1 illustrates the genotype-dependent effectiveness of sibutramine compared to placebo for GNB3 C825T polymorphism; and

FIG. 2 illustrates another view of the genotype-dependent effectiveness of sibutramine compared to placebo for GNB3 C825T polymorphism.

### DETAIL DESCRIPTION OF THE INVENTION

A placebo-controlled clinical study on the gene-dependent effectiveness of Sibutramine was carried out. This study included 105 patients with a body-mass index between 30 and 40 kg/m². All patients received a reduced calorie diet of maximum 1500-2000 kcal per day. Moreover the patients were urged to increase their physical activity and for example to walk at least 30 minutes a day at an increased pace. Forty nine patients received 15 mg Sibutramine daily to support weight reduction, while 56 patients received a placebo. The therapy was continued for 54 weeks. The body weight and body size of all patients were known at the beginning of the study and after 54 weeks of therapy. The end points of the study were the weight loss after 54 weeks, the number of patients with at least 5% reduction in body weight, and the number of patients with at least 10% reduction of body weight.

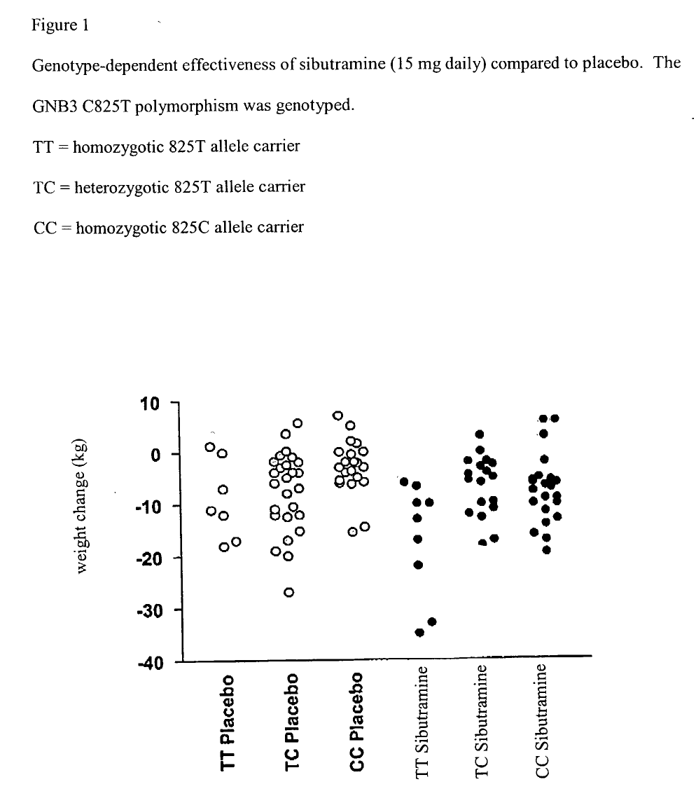

From all patients, DNA was isolated from a blood sample in order to analyze the C825T allele status in gene GNB3. Genotyping was done by means of PCR reaction and restriction analysis in processes familiar to one skilled in the area. The study results are shown in the graph of FIG. 1. In the placebo group, the average weight reduction was 5.3±7.3 kg, while the weight loss in the group treated with Sibutramine was 10.0±8.7 kg. This difference was statistically significant (p=0.036). FIG. 1 illustrates the genotype-dependent weight loss. In the placebo group, the average weight reduction was 9.1±2.8 kg for the TT genotype, 7.4±1.5 kg for the TC genotype, but only 2.8±1.1 kg for the CC genotype. This difference was statistically significant (One-way ANOVA, P=0.03). This result shows that non-pharmacological weight reduction measures, i.e, calorie restriction associated with increased physical activity, in 825T allele carriers leads to a reduction in body weight which is greater for persons with TT and TC genotypes than in persons with the CC genotype.

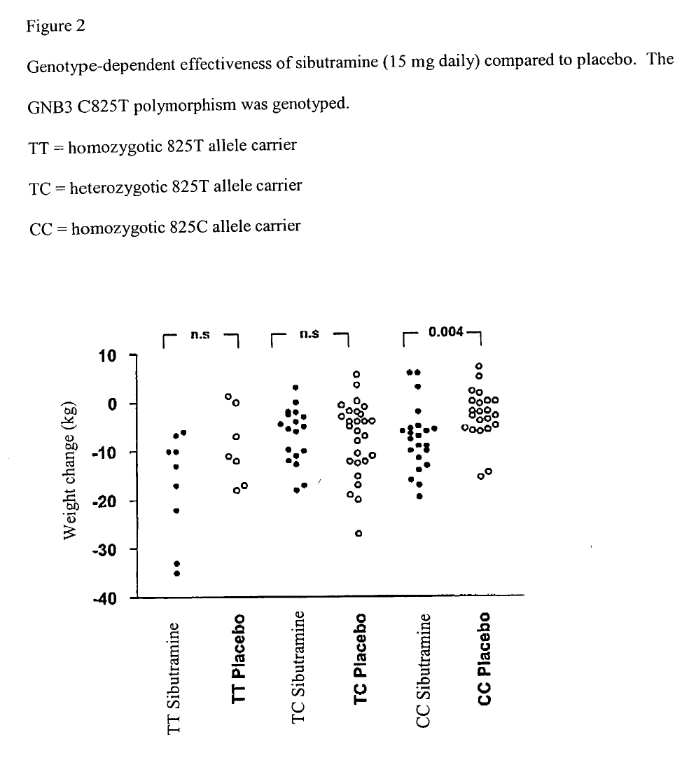

The combined comparison TC+TT versus CC genotype (graph in FIG. 1) also shows a clearly greater weight loss in 825T allele carriers. The same observation is made for the percentage weight loss. Here the 825T allele carriers also show increased weight reduction compared to the CC genotype (825C allele carrier). Interestingly, it is also observed in the groups treated with Sibutramine for the TT/TC genotype that there was no further weight loss compared to the placebo (see FIGS. 1-2). This means that the 825T allele carrier will not benefit from additional medication with Sibutramine.

Conversely, the strong effect of Sibutramine compared to the placebo is observed in individuals with the CC genotype. While individuals with the CC genotype (825C allele carrier) under a placebo lose only 2.6 kg, with Sibutramine they achieve a weight loss of 9.9 kg and can thus achieve the greater weight loss of 825T allele carriers (FIG. 2).

FIG. 2 shows the number of individuals within the genotype groups who lost more than 5% and more than 10% body weight with the placebo and Sibutramine. Furthermore, the long-term effect on weight loss is shown. All individuals were re-examined 2 years after the end of the study.

The numbers shown in FIG. 2 make it possible to compute the chance (odds ratio—OR) for homozygotic 825C allele carriers who lose more than 5% body weight in therapy with Sibutramine compared to the placebo. This odds ratio (OR) is computed to be (21/4)/(7/16); this corresponds to an OR of 12.0 (95% confidence interval 2.99-48.2). This OR is highly significant with a p value of 0.0002. The OR for the 10% weight reduction is 9.69 (1.8-50.4) at a p value of 0.0028. Conversely, the OR for 825T allele carriers is only 1.8 (p=0.26; not significant) and 1.6 (p=0.4) for a 5% and 10% weight loss. The same favorable results as for the 825C allele carrier were also found for the 3882A allele and the 5249G allele.

The long-term success after 2 years is especially surprising. For the CC genotype for a 5% weight reduction the OR is 4.4 (95% CI 1.038 to 19.02; p=0.0358) for a better effect of Sibutramine versus placebo. Conversely, the OR of 2.333 (0.6015 to 9.052) with a p value of 0.2139 is not significant.

These data clearly demonstrate that a gene change in gene GNB3 is outstandingly well suited for predicting the success of pharmacological and non-pharmacological weight reduction measures. In this example, the 825T allele carriers do not benefit from an adjuvant therapy with Sibutramine, while conversely patients with the CC genotype require pharmacologically defined additional therapy for weight reduction.

Thus it can be assumed that the effect of other anti-adiposity drugs can be predicted using a gene change in gene GNB3. These drugs include agonists on the β2 and β3 adrenoceptors, agonists on the leptin receptor, Amfepramon™, phenylpropanolamine, ephedrine, etc. Furthermore, centrally active drugs which reduce the appetite and feeling of hunger, drugs which delay emptying of the stomach and drugs which increase thermogenesis should be mentioned here.

As was shown here, the success of non-pharmacological measures can also be predicted. They include administration of a diet or certain diet products, structured weight reduction programs, such as Weight Watchers™, Optifast™, FormMed Concept™, BCM™ Concept, or therapeutic fasts.

The data presented here also establishes that, within the framework of weight reduction measures, 825T allele carriers and individuals with the 825 CC genotype experience different physiological, mainly hormonally-based, adaptation processes; due to reduced or increased activatability of G proteins. In this way, gene changes in gene GNB3 can be evaluated to characterize and understand the mechanism of action of weight-reducing measures. Thus, by using identification of a gene change in gene GNB3, the new development of weight reduction medications is also possible.

What is claimed is:

1. A method for determining the likelihood of success of pharmacological and non-pharmacological therapies for weight reduction comprising:
selecting a gene change in the human gene GNB3 which codes for the β3 subunit of G proteins,
relating the gene change to the success of at least one of a pharmacological and non-pharmacological therapy by conducting for each gene change the at least one pharmacological and non-pharmacological therapy for weight reduction in relation to a placebo, and
statistically determining the likelihood of success for each gene change of the at least one of a pharmacological and non-pharmacological therapy for a particular gene change based upon the weight reduction achieved.

2. The method as claimed in claim 1, wherein the pharmacological therapy includes the administration of a drug which is a reuptake inhibitor for serotonin or noradrenaline.

3. The method as claimed in claim 1, wherein the reuptake inhibitor is sibutramine.

4. The method as claimed in claim 1, wherein the pharmacological therapy includes the administration of a drug which reduces the resorption of dietary fat, increases lipolysis, or delays the emptying of the stomach.

5. The method as claimed in claim 1, wherein the pharmacological therapy includes the administration of an anti-adiposity drug selected from the group consisting of agonists on the leptin receptor, Amfepramon™, phenylpropanolamine, and ephedrine.

6. The method as claimed in claim 1, wherein the non-pharmacological therapy includes at least one of a reduction in daily caloric intake and an increase in daily exercise.

7. The method as claimed in claim 1, wherein the gene change is a C825T polymorphism in gene GNB3.
